(19) United States
(12) Patent Application Publication
(10) Pub. No.: US 2006/0040870 A1
(43) Pub. Date: Feb. 23, 2006

(54) Enhancement of apoptosis by caspase-9 inhibitors

(76) Inventors: Tucker W. LeBien, St. Paul, MN (US); Nisha Shah, Arden Hills, MN (US); Rebecca Asch, North Oaks, MN (US)

Correspondence Address: VIKSNINS HARRIS & PADYS PLLP, P.O. BOX 111098, ST. PAUL, MN, 55111-1098, US

(21) Appl. No.: 11/139,941

(22) Filed: May 27, 2005

Related U.S. Application Data
(60) Provisional application No. 60/575,529, filed on May 28, 2004.

(51) Int. Cl. A61K 38/06

(52) U.S. Cl. 514/18

(57) ABSTRACT

The present invention provides methods for inducing apoptosis in a tumor cell by contacting the cell with a caspase-9 inhibitor.

### CLAIM OF PRIORITY

This patent application claims priority to U.S. Application Ser. No. 60/575,529 filed on May 28, 2004. The instant application claims the benefit of the listed application, which is hereby incorporated by reference herein in its entirety.

### STATEMENT REGARDING FEDERALLY SPONSORED RESEARCH OR DEVELOPMENT

Funding for the work described herein was provided in part by the National Institutes of Health, grant number R01 CA31685. The federal government may have certain rights in the invention.

### FIELD OF THE INVENTION

This invention relates generally to methods and compositions for inducing apoptosis in tumor cells.

### RELATED TECHNOLOGY

Apoptosis is essential for the maintenance of tissue size and cell number homeostasis of multi-cellular organisms. Apoptotic abnormalities are thought to play an important role in the development of various neoplastic diseases as well as a number of neurodegenerative diseases.

Mitochondria play a key role in the regulation of apoptosis. A variety of important events in apoptosis involve mitochondria, including the release of caspase activators (such as cytochrome c), changes in electron transport, loss of mitochondrial transmembrane potential (which allows several proteins found within the mitochondrial intermembrane space to be liberated through the outer mitochondrial membrane, thereby participating in the apoptotic degradation phase), altered cellular oxidation-reduction, and which involves the of pro- and anti-apoptotic Bcl-2 family of proteins. The different signals that converge on mitochondria to trigger or inhibit these events and their downstream effects delineate several major pathways in physiological cell death.

### SUMMARY OF THE INVENTION

The present invention provides a method for inducing apoptosis in a tumor cell by contacting the tumor cell with a tetrapeptide apoptosis-inducing molecule. The tetrapeptide moiety is numbered P4-P3-P2-P1, wherein P2 is a histidine residue. As defined herein, the term “proapoptotic molecule” is a molecule that induces apoptosis, which is a process of cell death characterized by DNA cleavage, nuclear condensation and fragmentation, and plasma membrane blebbing that leads to phagocytosis of the cell without inducing an inflammatory response. Induction of apoptosis means that at least 10%, 20%, 30%, 40%, 50%, 60%, 70%, 80%, 90%, 95%, 99% or even 100% of the cells of a particular cell type that come into contact with the apoptosis-inducing molecule (or under particular growth conditions) undergo apoptosis. It should be noted that some molecules may be apoptosis-inducing for some cell types (or under certain growth conditions), and may be apoptosis-inhibiting for other cell types (or under other growth conditions). In certain embodiments, in the tetrapeptide apoptosis-inducing molecule, P1 is an aspartic acid residue, P2 is a histidine residue, P3 is a glutamic acid residue, and P4 is a leucine or tryptophan residue (i.e., Leu-Glu-His-Asp, SEQ ID NO:1 or Trp-Glu-His-Asp, SEQ ID NO:10).

The present invention provides methods for inducing apoptosis in a tumor cell by contacting the cell with an apoptosis-inducing molecule. A tumor cell can be exposed to an apoptosis-inducing molecule and induced to undergo apoptosis, either in the presence or the absence of other apoptosis-inducing compounds or conditions. Thus, apoptosis may be induced in the absence of other death-inducing stimuli. In addition, the tumor cell may lack caspase-9 activity.

The inhibitor can include a peptide moiety that allows for specific interaction with the active site of caspase-9. The inhibitor also may include a permeability-enhancing moiety (e.g., a hydrophobic moiety that facilitates entry of the inhibitor into the cell). In addition, the inhibitor may include a cross-linking moiety to promote irreversible cross-linking of the compound to the enzymatic active site of caspase-9.

The present invention provides methods for inducing apoptosis in a tumor cell by contacting the cell with a molecule that was previously identified and known in the art as a “caspase-9 inhibitor.”

The present invention further provides a pharmaceutical composition comprising a pharmaceutically acceptable carrier and an apoptosis-inducing molecule. The present invention also provides a method of treatment of leukemia or lymphoma in a patient, which method comprises administering to a patient a therapeutically effective amount of an apoptosis-inducing molecule.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

FIG. 1 is a series of histograms and contour plots showing levels of mitochondrial transmembrane potential (Δψm) and phosphatidylserine residues on the outer membrane of BLIN-3 and BLIN-4L cells that were treated with DMSO (D), C9i, and PCi for 2, 4, 6, and 8 hours, and stained with TMRE or Annexin V (AV). Numbers in the histograms indicate the percentage of PI⁻ cells that underwent a loss of Δψm. Numbers in the contour plots indicate the percentage of Annexin V⁺/PI⁻ cells.

FIG. 2 is a series of contour plots and histograms showing levels of cell permeability (YO-PRO) and Δψm in untreated BLIN-3 cells (FIBRO), and in BLIN-3 cells that were treated with DMSO, staurosporine (STSP), or the indicated concentrations (in TM) of C9i and PCi. Numbers in the contour plots indicate the percentage of YO⁻ PRO⁺/PI⁻ cells. Numbers in the histograms indicate the percentage of PI⁻ cells that underwent a loss of Δψm.

FIG. 3 is a series of graphs showing the sensitivity of the indicated BLIN cell lines to C9i in various culture conditions. Cells were cultured in medium alone, in medium supplemented with 10 ng/ml IL-7, on adherent fibroblasts, or on adherent fibroblasts supplemented with 10 ng/ml IL-7, as indicated in the figure legend. Cells were treated with DMSO, 50 μM C9i, or 50 TM PCi, as indicated below the groups of columns. Each column represents the mean of duplicate values.

FIG. 4 is a pair of graphs showing the effect of C9i on various cell lines, as determined by Annexin V staining and flow cytometry. The cells included the B-cell precursor ALL cell lines BLIN-1, BLIN-2, BLIN-3, BLIN-4E, BLIN-4L, and NALM-6, the RAMOS, RAJI, and DAUDI Burkitt lymphoma cell lines, the CCRF-CEM, JURKAT, and H9 T lymphoma cell lines, the K-562 erythroleukemia cell line, and resting or activated (i.e., CD3/CD28 cross-linked) normal human T cells. Each type of cell was cultured with DMSO, C9i, or PCi (upper panel), or DMSO, staurosporine, or staurosporine plus 50 μM C9i (lower panel). Data indicate the fold-increase in Annexin V⁺ cells. The light bars (P) in the upper panel represent the fold-increase in cells incubated with PCi compared to DMSO, and the dark bars (9) represent the fold-increase in cells incubated with C9i compared to DMSO. The light bars (ST) in the lower panel represent the fold-increase in cells incubated with staurosporine compared to DMSO, and the dark bars (9) represent the fold-increase in cells incubated with staurosporine plus C9i compared to DMSO.

FIG. 5 is a graph indicating the level of apoptosis in BLIN-4L cells that were electroporated with control or caspase-9 siRNA, cultured with DMSO, etoposide, or staurosporine, and stained with Annexin V. The etoposide and staurosporine values represent the percent of total Annexin V⁺ events (PI⁺ and PI⁻) minus the number present in DMSO, in order to eliminate the inherent difference in the apoptotic sensitivity of the two populations. The results represent the mean±standard deviation from three separate experiments. *, p<0.008 compared to control siRNA treatment (Wilcoxon Rank Sum method).

FIG. 6 is a chart showing the ability of various molecules, at different concentrations, to induce apoptosis in BLIN-1 cells.

FIG. 7 shows that C9i induces apoptosis in caspase-9-deficient cells.

### DETAILED DESCRIPTION OF THE INVENTION

The present invention relates to pharmaceutical compositions comprising compounds described herein and to their use as therapeutic agents, particularly in the treatment of cancer and cell proliferative disorders. More specifically, the compounds of this invention are useful in the treatment of a variety of cancers including, but not limited to pediatric acute lymphoblastic leukemia and adult non-Hodgkin's lymphoma.

The inhibitor can include a peptide moiety that allows for specific interaction with the active site of caspase-9. The inhibitor also may include a permeability-enhancing moiety (e.g., a hydrophobic moiety that facilitates entry of the inhibitor into the cell). In addition, the inhibitor may include a cross-linking moiety to promote irreversible cross-linking of the compound to caspase-9.

Peptide Moiety

In certain embodiments, the caspase-9 inhibitor can be Z-LEHD-fmk (also referred to herein as C9i (“LEHD” is Leu-Glu-His-Asp, SEQ ID NO:1). In some embodiments, the caspase-9 inhibitor can be a variant of Z-LEHD-fmk. For example, the peptide moiety can have the amino acid sequence Ala-Glu-His-Asp (AEHD, SEQ ID NO:2), Leu-Glu-Ala-Asp (LEAA, SEQ ID NO:3), Leu-Glu-His-Asp (LEHA, SEQ ID NO:4), Leu-Glu-Glu-Asp (LEED, SEQ ID NO:5), Leu-Glu-Ala-Asp (LEAD, SEQ ID NO:6), Leu-Glu-Lys-Asp (LEKD, SEQ ID NO:7), Leu-Glu-Arg-Asp (LERD, SEQ ID NO:8), Leu-Ala-His-Asp (LAHD, SEQ ID NO:9), Trp-Glu-His-Asp (WEHD, SEQ ID NO:10), Ala-Glu-His-Asp (AEHD, SEQ ID NO:11), or Phe-Glu-His-Asp (FEHD, SEQ ID NO:12). In certain embodiments, the peptide may be a trimer, such as Glu-His-Asp (EHD, SEQ ID NO:13), rather than a tetramer. In certain embodiments, the peptide moiety is WEHD or AEHD.

Permeability-Enhancing Moiety

The inhibitor can have a permeability-enhancing moiety such as a benzoxycarbonyl group. Alternatively, the caspase-9 inhibitor can lack a permeability-enhancing moiety.

Cross-Linking Moiety

The inhibitor can have a cross-linking moiety such as fluoromethyl ketone. Alternatively, the caspase-9 inhibitor can lack a cross-linking moiety and may be attached to an aldehyde (CHO) group.

Inhibitors

In one embodiment, the permeability-enhancing moiety is a benzoxycarbonyl group, the peptide moiety is a tetramer with the amino acid sequence Leu-Glu-His-Asp (SEQ ID NO:1), and the cross-linking moiety is fluoromethyl ketone. In another embodiment, the permeability-enhancing moiety is a benzoxycarbonyl group, the peptide moiety is a tetramer with the amino acid sequence Trp-Glu-His-Asp (SEQ ID NO:10), and the cross-linking moiety is fluoromethyl ketone.

Formulations of Inhibitors

The caspase-9 inhibitor compounds of the present invention can be formulated as pharmaceutical compositions and administered to a mammalian host, such as a human patient in a variety of forms adapted to the chosen route of administration, that is, orally or parenterally, by intravenous, intramuscular, topical or subcutaneous routes.

Thus, the present compounds may be systemically administered, for example, orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of the invention to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of the invention can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compound(s) of the invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg per day, e.g., from about 1 to about 60 mg/kg of body weight per day or about 2 to 50 mg/kg per day.

The compound may conveniently be administered in unit dosage form; for example, containing 5 to 1,000 mg, conveniently 10 to 750 mg, most conveniently, 50 to 500 mg of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

### EXAMPLE 1

### Enhancement of Stress-Induced Apoptosis in B-Lineage Cells By Caspase-9 Inhibitor

Normal lymphoid cell development in the bone marrow and thymus is autonomously regulated by transcription factors that modify patterns of gene expression (Glimcher Cell. 1999;96:13-23) and externally regulated by the extracellular matrix, stromal cells, and cytokines/chemokines/colony-stimulating factors (Anderson Semin Immunol. 2000;12:457-464; Bertrand Immunol Rev. 2000;175:175-186). The fate of developing lymphoid cells is further influenced by the functional balance of pro- and anti-apoptotic proteins expressed at specific points in development, and the survival signals that are transduced by external cues such as cytokines (Opferman Nat Immunol. 2003;4:410-415; Marsden Annu Rev Immunol. 2003;21:71-105).

Induction of apoptosis proceeds through two main pathways. The first initiates at cell surface death receptors such as CD95/Fas or the tumor necrosis receptor (the extrinsic pathway) (Krammer Nature. 2000;407:789-795; Locksley Cell. 2001;104:487-501). The second is triggered by stress, genotoxic agents or cytokine deprivation (the intrinsic or mitochondrial pathway) (Wang Genes Dev. 2001;15:2922-2933; Newmeyer Cell. 2003;112:481-490). The biochemical coupling of pro-apoptotic molecules of the BH3 death domain and Bcl-2 family members to caspase activation and downstream dismantling of cellular structure has been extensively studied in all metazoans. However, caspase activation by itself does not guarantee that apoptotic signals proceed unchecked. Members of the inhibitor of apoptosis (IAP) family of proteins can suppress or delay apoptosis by inhibiting the enzymatic activity of initiator and executioner caspases (Salvesen Nat Rev Mol Cell Biol. 2002;3:401-410; Shi Mol Cell. 2002;9:459-470). Whereas activation of initiator (e.g., caspases-8, 9 and 10) and executioner (e.g., caspases-3, 6 and 7) caspases generally occur by oligomerization of monomeric zymogens and caspase-mediated cleavage, respectively (Boatright Mol Cell. 2003;11:529-541; Donepudi Mol Cell. 2003;11:543-549; Thornberry Science. 1998;281:1312-1316), the consequences of inhibition of activated caspases are not completely understood. Although activation of executioner caspases leads to irreversible commitment to death when IAP function is neutralized by SMAC/DIABLO, loss of mitochondrial function does not. Outer mitochondrial membrane permeabilization (and concomitant translocation of cytochrome C) is not an irreversible event, since caspase inhibition at this juncture can lead to restoration of mitochondrial transmembrane potential (Δψm) (Waterhouse J Cell Biol. 2001;153:319-328). This result might be explained by the recent finding that caspase translocation into the mitochondria leads to cleavage of electron transport complexes I and II and further dissolution of mitochondrial integrity (Ricci J Cell Biol. 2003;160:65-75).

Mammalian B-cell development is an excellent model to study cell fate decisions that culminate in survival or death, since functional immunoglobulin gene rearrangement and appropriate cues from the bone marrow (BM) microenvironment are essential for survival and differentiation (LeBien Blood. 2000;96:9-23). Perturbations in the balance between pro and anti-apoptotic signals can contribute to development of lymphohematopoietic malignancies (Cory Nat Rev Cancer. 2002;2:647-656). During the course of characterizing the role of the mitochondrial pathway in the demise of bone marrow stromal cell-dependent B-lineage acute lymphoblastic leukemia (ALL), the inventors made the unexpected observation that a selective inhibitor of caspase-9 (Z-LEHD-fmk) enhanced (rather than retarded) apoptosis to selected stimuli. They further demonstrated that this inhibitor also induced apoptosis in healthy B-lineage ALL cells and several other leukemia/lymphoma cell lines. Thus, a four amino acid peptide previously identified by its capacity to inhibit caspase-9 activity also acted as a pro-apoptotic peptide.

Materials and Methods

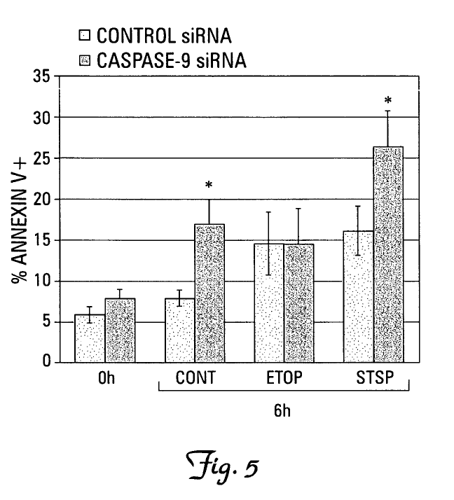

Origin and maintenance of cell lines. Establishment and characterization of the BM stromal cell-dependent B-lineage ALL cell lines BLIN-2, BLIN-3 and BLIN-4L have been previously described (Shah Blood. 1998;92:3817-3828; Bertrand Blood. 2001;98:3398-3405; Shah Cancer Res. 2001;61:5268-5274). The three cell lines were maintained on non-irradiated human foreskin fibroblasts in X-VIVO 10 serum-free medium (Bio-Whittaker, Walkersville, Md.). Proliferation of BLIN-3 and BLIN-4L was enhanced by addition of 1 ng/ml recombinant human IL-7 (PeproTech, Rocky Hill, N.J.). The leukemic cells were removed by gentle washing of the fibroblast monolayers and were >90% viable prior to being utilized in individual experiments. All other leukemia and lymphoma cell lines (FIG. 5) were maintained in RPMI-1640 supplemented with 10% fetal bovine serum and were tested for Mycoplasma infectivity bi-monthly.

Normal B-lineage cells. CD19+ B-lineage cells were derived from CD34+ human cord blood stem cells plated on the murine stromal cell line MS-5 (Itoh Exp Hematol. 1989;17:145-153). MS-5 stromal cells were kindly provided by Dr. Paul Kincade (University of Oklahoma Medical Research Foundation), following permission from Professor K. John Mori (Niigata University, Japan). FACS-purified CD34+/CD38+/lineage—cord blood hematopoietic stem cells were plated onto confluent, non-irradiated MS-5 stromal cells in 96-well flat-bottom plates (200 cells/well) containing Minimal Essential Medium supplemented with 10% FBS. Stem cell factor and G-CSF were added at 10 ng/ml (twice weekly) to enhance the emergence of CD19+ B-lineage cells (Nishihara Eur J Immunol. 1998;28:855-864; Ohkawara Leukemia. 1998;12:764-771). Analysis of cultures at approximately 4 weeks revealed that 10 to 30% of cells with lymphoid light scatter characteristics were CD19+. Wells were then pooled, stained with PE-conjugated anti-CD 19 + propidium iodide (PI), and sorted on a FACSVantage (BD Immunocytometry Systems, San Jose, Calif.). Cord blood CD34+/CD38+/lineage—cells were provided by Valerie McCullar and Dr. Jeff Miller of the University of Minnesota Cancer Center in accordance with the University of Minnesota Institutional Review Board on Human Subjects.

Chemicals and reagents. Caspase inhibitors were purchased from R&D Systems (Minneapolis, Minn.) and Calbiochem (La Jolla, Calif.). Stock solutions were prepared to 20 mM in DMSO and stored at −20° F. The following irreversible inhibitors were used: caspase-2 (Z-VDVAD-fmk), caspase-3 (Z-DEVD-fmk and Z-DQMD-fmk), caspase-6 (Z-VEID-fmk), caspase-8 (Z-IETD-fmk), caspase-9 (Z-LEHD-fmk) and pan-caspase (Z-VAD-fmk). The inventors also used the reversible caspase-9 inhibitor LEHD-CHO (cat# P446, Biomol Research Laboratories, Plymouth Meeting, Pa.). The protein kinase C/serine-threonine kinase inhibitor staurosporine was purchased from Alexis Biochemicals (San Diego, Calif.).

Flow cytometry. Quantitation of apoptotic events was conducted using a FACS Calibur (BD Immunocytometry Systems). All analyses were conducted on cells with lymphoid light scatter characteristics (i.e., low to medium forward scatter and low 90° side scatter).

i. Mitochondrial transmembrane potential (Δψm). The cationic lipophilic fluorescent dye tetramethylrhodamine, ethyl ester, perchlorate (TMRE) was purchased from Molecular Probes, Inc. (Eugene, Oreg.). TMRE has a high degree of membrane permeability and accumulates into mitochondria in accord with the Nernstian equation (Farkas Biophys J. 1989;56:1053-1069). Cells were washed once and re-suspended to 0.5-1.0×10⁶/ml in X-VIVO 10. TMRE was added to a final concentration of 50 nM and the cells were incubated in the dark at 37° C. for 15 minutes. Cells were then washed 2× in PBS, resuspended in PBS containing 2 μg/ml of propidium iodide (PI), and immediately analyzed. The protonophore carbonylphenylhydrazone (50 μM, CCCP, Sigma) was used as a positive control to effect loss of Δψm in 100% of cells.

ii. YO-PRO-1. The green fluorescent DNA intercalant dye YO-PRO-1 (Idziorek J Immunol Methods. 1995;185:249-258) was purchased from Molecular Probes as the Vybrant Apoptosis Assay Kit #4. Dual staining with YO-PRO and PI allows for the detection of early apoptotic cells (YO-PRO+/PI−) that have undergone initial changes in permeability to small molecules. Cells were stained according to the manufacturers recommendations.

iii. Annexin V. FITC-labeled Annexin V (BD Pharmingen, San Diego, Calif.) was used to detect external phosphatidylserine residues appearing on the external surface of early apoptotic cells (Koopman Blood. 1994;84:1415-1420). Cells were stained according to the manufacturers recommendations.

General experimental strategy. BLIN cell lines were removed from fibroblast monolayers, washed once and resuspended to 7.5×10⁵/ml in X-VIVO 10. Two hundred μL of resuspended cells were added to 96-well flat-bottom microtiter plates (ISC Bioexpress, Kaysville, Vt.), followed by 0.5-1.0 μL of caspase inhibitors in DMSO. An identical volume of DMSO alone was added to separate wells as a vehicle control. This final DMSO concentration of 0.25-0.5% vol/vol had no adverse affect on any of the BLIN cell lines. At the indicated times the entire content of individual wells was harvested and stained in the fluorescent protocols described above.

RNA interference using small interfering (si) RNA Specific suppression of caspase-9 expression was accomplished using siRNAs (Elbashir Nature 2001;411:494-498). The Dharmacon Inc. (Lafayette, Colo.) SMARTpool design technology was used to target human caspase-9. This technology employs a double algorithm. The first is designated SMARTselection and uses 33 criteria and parameters that effectively eliminate non-functional siRNAs. The second identifies four siRNA duplexes from the original SMARTselected pool. In preliminary studies using FITC-conjugated control siRNA (Dharmacon), we determined that the BLIN cell lines could be transfected to >80% efficiency by electroporation. Based on these results the following protocol was developed. BLIN-4L cells were washed and adjusted to 2.5×10⁷ cells/ml in Opti-MEM I (cat # 31985-070, Invitrogen, Carlsbad, Calif.). Two hundred μL of target cells were then added to Gene Pulsar Cuvettes (cat # 165-2088, BioRad, Hercules, Calif.) and electroporation was conducted using a BTX Electroporation System/ElectroSquare Porator T820. Cells received a single pulse of 240 volts and were rested for 30 minutes at room temperature. BLIN-4L were then diluted into 4.8 ml of X-VIVO 10 supplemented with 10% FBS and 5 ng/ml of IL-7. The 5 ml suspension was then cultured for 3 days in 25 cm² flasks to effect reduction of caspase-9 protein prior to testing in apoptotic assays. This was sufficient to promote recovery (i.e., maintain survival) of the cells in the absence of significant proliferation (<2-fold increase in cell number in 3 days).

Western blotting. This method was conducted essentially as previously described.²⁰ The antibodies used were: rabbit anti-caspase-2 (catalog # sc-625, Santa Cruz Biotechnology Inc., Santa Cruz, Calif.), rabbit anti-caspase-3 (catalog # 9661, Upstate Cell Signaling Solutions, Waltham, Mass.), rabbit anti-caspase-6 (catalog # 9761, Upstate), rabbit anti-caspase-9 (catalog # 9502, Upstate), mouse anti-PARP (catalog # 556494, Pharmingen), and rabbit anti-Bid (catalog # AF846, R&D Systems). The antibodies to caspase-2 and caspase-9 were generated to the procaspase isoforms, whereas the antibodies to caspase-3 and caspase-6 were generated to cleavage products.

Results

Apoptosis of B-lineage ALL cells is enhanced by an irreversible inhibitor of caspase-9. The apoptotic pathway activated in B-lineage ALL cell lines deprived of their adherent cell microenvironment includes loss of Δψm (detected by TMRE staining), appearance of phosphatidylserine residues on the outer membrane (detected by Annexin V staining), and cleavage of procaspases 2, 3, 6 and 9 (refs. 19-21 and data not shown). Since these results implicate the mitochondrial/apoptosome pathway in the demise of BLIN cell lines, the inventors determined whether the caspase-9 inhibitor (C9i) Z-LEHD-fmk would delay cell death. Remarkably, the opposite result was obtained.

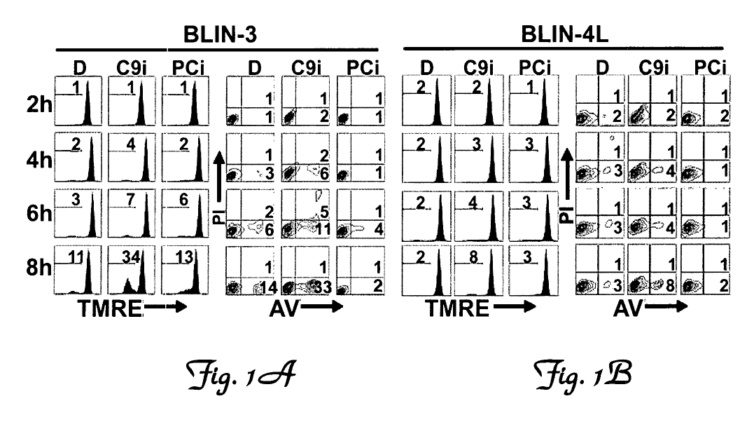

A typical experiment is shown in FIG. 1. BLIN-3 and BLIN-4L cells were removed from adherent fibroblasts, incubated with 50 μM C9i, and assayed by Annexin V and TMRE staining at 2-hour time points. Incubation of BLIN-3 with C9i led to pronounced enhancement of apoptosis in BLIN-3 beyond that which occurred in control cells incubated with the DMSO vehicle alone. Inclusion of 50 μM PCi did not prevent loss of Δψm in BLIN-3 (compare results to DMSO), suggesting that the initial apoptotic events leading to loss of mitochondrial function are minimally caspase-dependent. However, PCi completely blocked the appearance of Annexin V+ cells, consistent with the effector caspase-dependency of this characteristic of apoptosis. C9i also promoted apoptosis in BLIN-4L, albeit not as pronounced as in BLIN-3. In other experiments with BLIN-4L cells, the level of apoptotic enhancement by C9i was twice that shown in FIG. 1. BLIN-3 cells and BLIN-4L cells cultured in medium alone or in medium supplemented with DMSO (the latter shown in FIG. 1) gave identical results. This precluded any contribution of DMSO to the induction of apoptosis by C9i.

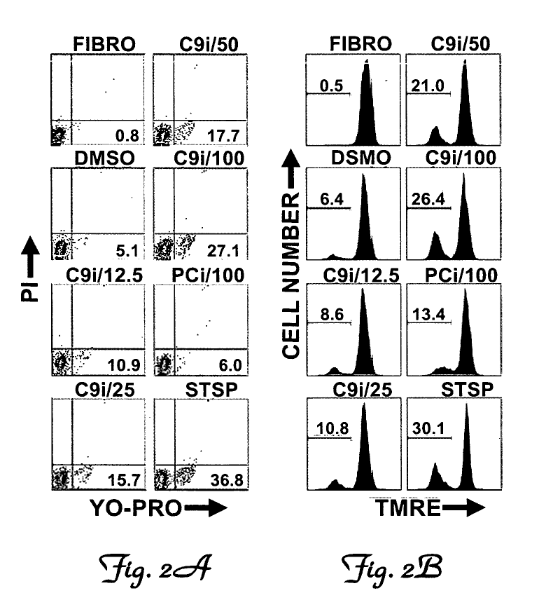

As shown in FIG. 2, enhancement of apoptosis by C9i was dose-dependent. The percentage of cells in a given condition that were YO-PRO+/PI− was similar to the percentage of cells with reduced TMRE staining. This result probably reflects a concomitant change in cell membrane permeability and loss of Δψm.

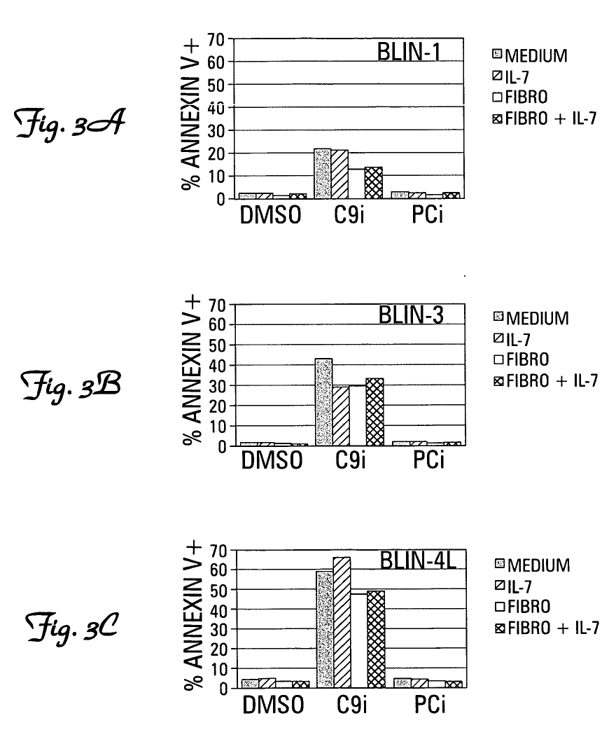

Overnight incubation of BLIN-3 and BLIN-4L cells with 50 μM C9i led to cleavage of the caspase-3/caspase-7 substrate PARP. As expected, inclusion of 50 μM PCi had no effect. The degree of PARP cleavage in the presence of C9i (greater in BLIN-3 than BLIN-4L) correlated with the percentage of Annexin V+ cells (FIG. 3). In other experiments the inventors demonstrated that: a) irreversible inhibitors of caspases 2, 3, 6 and 8 did not enhance apoptosis, b) C9i enhancement of apoptosis occurred at concentrations as low as 6.25 μM, c) identical results were obtained using C9i from two separate vendors, and d) the inventors could confirm the capacity of C9i to block apoptosis at some level since 50 μM C9i blocked the Fas response of JURKAT T lymphoma cells.

Effect of C9i on normal B-cell precursors. Normal CD19+ B-cell precursors and CD33+ myeloid cells were derived by plating of CD34+/CD38+/lin—cord blood hematopoietic stem cells onto murine MS-5 stromal cells. After 4 weeks the CD19+ and CD33+ cells were purified by cell sorting and tested for their sensitivity to C9i. Table 1 shows one of three separate experiments that gave similar results. Consistent with the results obtained using the stromal cell dependent BLIN cell lines, C9i significantly enhanced the death of CD19+ cells removed from their MS-5 microenvironment, whereas PCi partially inhibited death. In contrast, CD33+ myeloid cell death was partially inhibited by C9i and PCi.

TABLE 1
Effect of caspase-9 inhibitor on lymphoid and myeloid progenitors
derived from cord blood hematopoietic stem cells.

| | | Treatment | | |
|---|---|---|---|---|
| Cell Type | Time 0 | DMSO | C9i | PCi |
| CD19⁺ | 1.5 | 46.5 ± 1.4 | 56.0 ± 3.2* | 39.5 ± 1.8* |
| CD33⁺ | <10 | 27.9 ± 3.9 | 22.3 ± 1.2 | 18.0 ± 3.9* |

CD19+/PI− and CD33⁺(ie.CD19⁻)/PI− cells were sorted and tested for sensitivity to C9i as described in the Materials and Methods. Cells were cultured in triplicate with DMSO, C9i or PCi, harvested after 18 hours; and stained with Annexin V.
Values are mean ± SD of the percentage of total Annexin V+ cells (PI+ and PI−).
p < 0.05 compared to DMSO control by students t test

Differential effect of C9i on other leukemic cells. In preliminary experiments the inventors found that C9i did not promote apoptosis in BLIN-3 and BLIN-4L when the cells were maintained on adherent fibroblasts and/or IL-7 for 8 hours. However, when the incubation period was extended to 18 hours, C9i promoted apoptosis in BLIN-3 and BLIN-4L maintained on adherent fibroblasts and/or IL-7 (FIG. 3). This result suggested that C9i could also promote apoptosis in non-stressed, healthy cells.

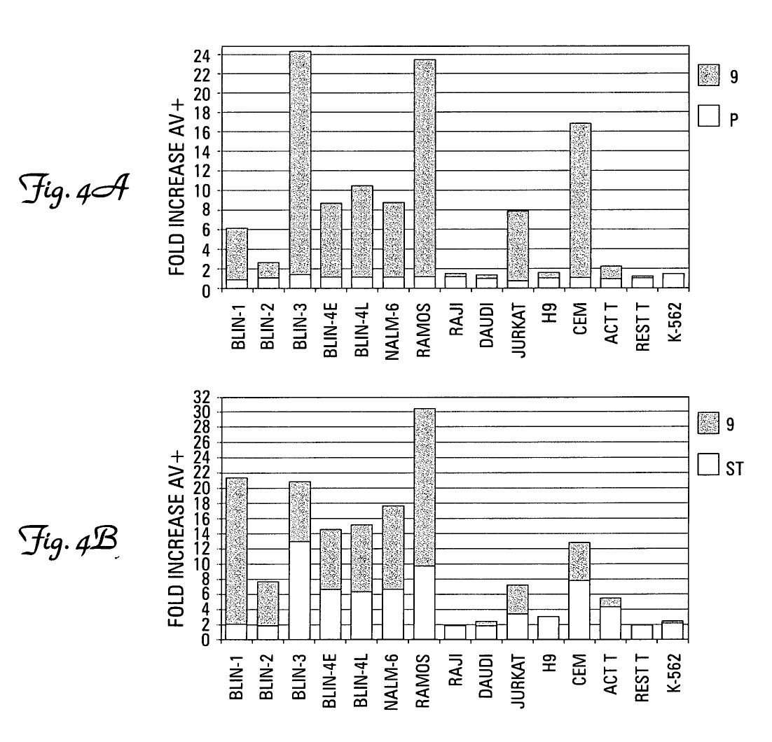

The inventors expanded their analysis of C9i to other leukemic cell lines. As shown in FIG. 4, incubation of a panel of leukemic cell lines with 50 μM C9i for 18 hours revealed a wide range of sensitivities. All six B-cell precursor ALL cell lines (BLIN-1, 2, 3, 4E, 4L and NALM-6) were C9i sensitive, although BLIN-2 was less sensitive than the others. In addition, the RAMOS Burkitt lymphoma and the CCRF-CEM T lymphoma cell lines were extremely sensitive. The JURKAT T lymphoma cell line was also sensitive. In contrast, the RAJI and DAUDI Burkitt lymphoma cell lines, the H9 T lymphoma cell line, and the K-562 erythroleukemia cell line were insensitive to C9i. Resting and activated (i.e., CD3/CD28 cross-linked) normal human T cells were also analyzed, with activated T cells being marginally sensitive. These collective results indicate that leukemic (and normal, Table 1) B-cell precursors are uniformly sensitive to C9i, with other leukemia/lymphoma cell lines showing a wide variation in sensitivity. The inventors also tested whether C9i would potentiate the apoptotic effect of the widely used serine/threonine kinase inhibitor staurosporine. As shown in FIG. 4, C9i enhanced the staurosporine response of those leukemic cells that responded to C9i alone. Thus, the leukemic cell lines were generally responsive to both C9i and staurosporine, or responsive to neither.

Apoptotic sensitivity of cells made deficient in caspase-9 by siRNA. To determine whether the results obtained with C9i could be corroborated by another method, the inventors turned to RNA interference using siRNA. A SMARTpool of siRNA specific for human caspase-9 effectively and specifically suppressed-BLIN-4L caspase-9 protein in a dose-dependent manner. In contrast, an identical amount of control siRNA had no effect on caspase-9. This degree of caspase-9 inhibition was reproducibly achieved 48-72 hours after electroporation.

The inventors then tested whether caspase-9 deficient BLIN-4L would undergo a heightened response to apoptotic stimuli. As shown in FIG. 5, caspase-9 deficient BLIN-4L were significantly more sensitive to staurosporine than control siRNA treated cells. In contrast, there was no difference in the response to etoposide. It is noteworthy that caspase-9 deficient cells incubated in medium alone also underwent a significantly greater degree of apoptosis than control siRNA treated cells.

Discussion

The interactions between normal and leukemic B-lineage cells and BM stromal cells are bi-directional. BM stromal cells transduce signals to B-lineage cells (Gibson Leuk Lymphoma. 2002;43:19-27), and contact of human B-lineage cells with BM stromal cells induces phosphorylation of several substrates and synthesis of IL-6 in stromal cells (Jarvis J Immunol. 1995;155:2359-2368; Jarvis Blood. 1997;90:1626-1635). Apoptotic pathways activated in B-lineage ALL following loss of stromal cell contact can be considered a stress response, or death by neglect. However, apoptotic pathways activated in this example of heterotypic cellular dependency are not necessarily identical to the pathways activated by DNA-damaging agents (e.g., x-irradiation or chemotherapeutic drugs) or single cytokine deprivation (e.g., IL-3 or IL-7). Death by neglect in vivo may be initiated when leukemic cell crowding around a stromal cell niche leads to an increase in leukemic cells lacking direct contact with stromal cell membranes. Failure of a leukemic cell to have access to a stromal cell niche could then activate an apoptotic cascade, or trigger further mutations that lead to the emergence of stromal cell independent subclones.

BLIN cell lines deprived of fibroblast monolayers undergo a gradual accumulation of apoptotic cells culminating in death within 24-48 hours (Shah Blood. 1998;92:3817-3828; Bertrand Blood. 2001;98:3398-3405; Shah Cancer Res. 2001;61:5268-5274). Since caspase-9 activation generally occurs after a loss in mitochondrial function, C9i would not be expected to inhibit loss of Δψm. However, it was unexpected to discover that C9i enhanced the loss of Δψm (FIGS. 1 and 2) and concomitant or subsequent events downstream of mitochondrial function, including Annexin V binding (FIG. 1) and membrane permeability changes (FIG. 2). The C9i effect was dose-dependent (FIG. 2) and could be detected at concentrations as low as 6.25 μM. Enhancement of apoptosis was unique to treatment with C9i. Inhibitors of caspase-2, caspase-3, caspase-6, caspase-8 and the pan-caspase inhibitor Z-VAD-fmk did not enhance apoptosis. Western blot results corroborated the flow cytometry data in FIGS. 1 and 2 and demonstrated that C9i treatment led to PARP cleavage; an essentially universal response to caspase-3/caspase-7 activation. The inventors also detected activation of several caspases (i.e., caspases-2, 3, 6 and 9) in C9i treated cells (data not shown). Thus, independent of mechanism, simple treatment of BLIN-3 and BLIN-4L with 50 μM C9i culminates in the activation of a caspase cascade.

The C9i enhancement of apoptosis occurred in all of the tested adherent cell-dependent BLIN cell lines, and to a lesser degree in normal adherent cell-dependent CD19+ B-lineage cells derived from cord blood CD34+ hematopoietic stem cells (Table 1). Thus, the enhanced apoptotic pathway is not unique to leukemic B-lineage cells. Moreover, C9i did not enhance apoptosis of normal stromal cell-dependent CD33+ myeloid cells and actually had a minor inhibitory effect (Table 1). The latter is an important control since it confirms the capacity of C9i to inhibit apoptosis of normal lymphohematopoietic (largely myeloid) cells that emerge under the same conditions as the CD19+ B-lineage cells.

Experiments examining the sensitivity of healthy leukemic cell lines under the conditions shown in FIG. 1 indicated that C9i did not induce apoptosis (data not shown). Further analysis revealed that healthy cells incubated for 18-24 hours with 50 μM C9i underwent varying degrees of apoptosis (FIGS. 3 and 4). Notably, BLIN-3 and BLIN-4L treated with C9i and incubated on fibroblast monolayers and/or IL-7 were still C9i sensitive (FIG. 3). Thus, C9i is not simply promoting stress-induced apoptosis. FIG. 4 shows that not all cells were sensitive, and there was an excellent correlation between C9i and staurosporine sensitivity at the level of individual leukemic cell lines. The fact that some healthy leukemic cell lines are C9i sensitive (e.g., RAMOS and CEM) indicates that C9i is exerting a proapoptotic effect that is functionally the opposite of its simple inhibition of activated caspase-9. It is important to emphasize that irreversible inhibitors for caspase-2, caspase-3, caspase-6 and caspase-8 did not promote apoptosis, as would be expected. Since all inhibitors are synthesized with a benzyloxycarbonyl group (the Z group) at the N-terminus and a fluoromethyl ketone group (fmk) at the C-terminus, these chemical modifications could not confer apoptotic properties on C9i. Rather, there must be something unique about the LEHD sequence that activates an apoptotic pathway. One clue may lie in the fact that loss of Δψm is an early consequence of C9i treatment. Thus, C9i could be acting at the level of the outer mitochondrial membrane, or could be exerting an effect comparable to activation of a BH3 death domain.

Given the limitations of using a short peptide inhibitor such as C9i, the inventors used RNA interference to more directly assess the role of caspase-9. They developed a protocol that consistently led to >90% reduction in caspase-9 protein expression. They further demonstrated that BLIN-4L cells deficient in caspase-9 exhibited an enhanced response to staurosporine (FIG. 5), consistent with the results obtained with C9i (FIG. 4). Caspase-9 deficient BLIN-4L also exhibited a significantly enhanced level of apoptosis following 6-hour removal from adherent fibroblasts.

In conclusion, the inventors have shown through use of the caspase inhibitor Z-LEHD-fmk and targeting of caspase-9 by RNA interference, that B-lineage ALL undergo heightened spontaneous and drug-induced apoptosis. The sensitivity of B-lineage ALL and some other leukemia/lymphoma cell lines to C9i alone suggests that it transduces a proapoptotic signal in some cells.

### EXAMPLE 2

### Influence of Specific Amino Acid Substitutions on the Pro-Apoptotic Activity of Caspase-9 Inhibitor

Caspases are constitutively express in cells, and are usually in their pro-form. In response to an incident, such as irradiation or chemotherapy, the pro-form of the caspase is converted into the activated state. Certain compounds are known that can inhibit caspases. For example, the pan-caspase (PCi) VAD can block all caspases. Other compounds inhibit only certain caspases. For example, C2i inhibits caspase-2. This particular inhibitor is very dose dependent. The sequences of certain caspase inhibitors, and variants of one of the inhibitors, are provided in the table below.

TABLE 2

| | | Pro-apoptotic Activity |
|---|---|---|
| Caspase Inhibitor Sequences | | (after 24 hours) |
| PCi | VAD | − |
| C2i | VDVAD | − |
| C3i | DEVD | − |
| C6i | VEID | − |
| C8i | IETD | −− |
| C9i | LEHD | ++++ |
| C9i/D > A | LEHA | ND |
| C9i/H > E^{c} | LEED | − |
| C9i/H > A | LEAD | − |
| C9i/H > K | LEKD | ND |
| C9i/H > R | LERD | ND |
| C9i/E > A | LAHD | ND |
| C9i/L > W^{d} | WEHD | ++++ |
| C9i/L > A | AEHD | ++ |
| C9i/L > F | FEHD | ND |
| C9i/ΔL | EHD | ND |
| C91/ΔFMK | LEHD^{e} | − |

^{a}All inhibitors harbor a irreversible chemical modification, where Z = benzyloxycarbonyl and FMK = fluoromethylketone. The “Z” moiety promotes cellular uptake of the inhibitor. The “FMK” moiety irreversibly cross-links the inhibitor to the active site of the caspase. The underlined amino acid residue indicates substitutions distinct from the LEHD parent compound.
^{b}Pro-apoptotic activity against leukemia/lymphoma cells, where “−” is no activity, ++++ is maximum activity, and ND is not determined.
^{c}This sequence is marketed as exhibiting selective inhibition of caspase-13.
^{d}This sequence is marketed as exhibiting selective inhibition of caspase-1.
^{e}The FMK moiety is removed.

The inventors carried out experiments to test the influence of specific amino acid substitutions on the proapoptotic activity Z-LEHD-fmk. Cells were cultured for 24 hours with various μM concentrations of Z-LEDH-fmk or single amino acid substitution of the parent peptide. All values in the following Table 3 represent the percentage of total Annexin V+ cells (PI+ and PI−) minus the percentage present in culture with DMSO alone (BLIN-2=3%; BLIN-3+7%; BLIN4L=5%).

TABLE 3
INFLUENCE OF SPECIFIC AMINO ACID SUBSTITUTIONS
ON THE PROAPOPTOTIC ACTIVITY OF Z-LEHD-FMK.

| | [symbol] | | | | [symbol] | | | | [symbol] | | | |
|---|---|---|---|---|---|---|---|---|---|---|---|---|
| | 12.5 | 25 | 50 | 100 | 12.5 | 25 | 50 | 100 | 12.5 | 25 | 50 | 100 |
| BLIN-1 | 22 | 24 | 66 | 45 | 17 | 23 | 33 | 62 | 2 | 3 | 3 | 7 |
| BLIN-2 | 24 | 35 | 52 | 46 | 18 | 13 | 19 | 40 | 2 | 1 | 4 | 50 |
| BLIN-3 | 59 | 88 | 91 | 86 | 34 | 28 | 48 | 51 | 6 | 12 | 32 | 78 |
| BLIN-4L | 19 | 25 | 33 | 16 | 6 | 10 | 12 | 36 | 3 | 0 | 1 | 11 |
| RAMOS | 83 | 96 | 91 | 84 | 85 | 74 | 86 | 99 | 6 | 8 | 11 | 41 |

| | |
|---|---|
| [symbol] | [symbol] |

| | 12.5 | 25 | 50 | 100 | 12.5 | 25 | 50 | 100 |
|---|---|---|---|---|---|---|---|---|
| BLIN-1 | 2 | 2 | 2 | 4 | 2 | 3 | 4 | 3 |
| BLIN-2 | 1 | 2 | 1 | 2 | 2 | 2 | 1 | 2 |
| BLIN-3 | 2 | 4 | 7 | 11 | 3 | 6 | 9 | 12 |
| BLIN-4L | 0 | 0 | 0 | 3 | 0 | 0 | 0 | 2 |
| RAMOS | 6 | 8 | 8 | 17 | 5 | 6 | 7 | 14 |

Circles indicate specific amino acid substitutions compared to parent peptide LEHD.

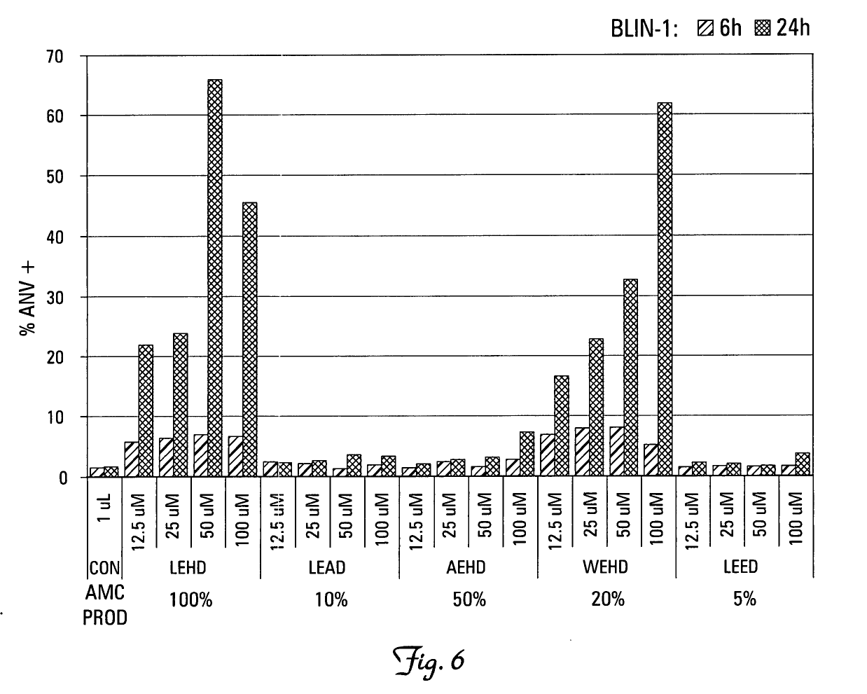

The inventors also tested the impact of various concentrations of the parent and modified inhibitors on BLIN-1 cells after either 6 or 24 hours. (FIG. 6)

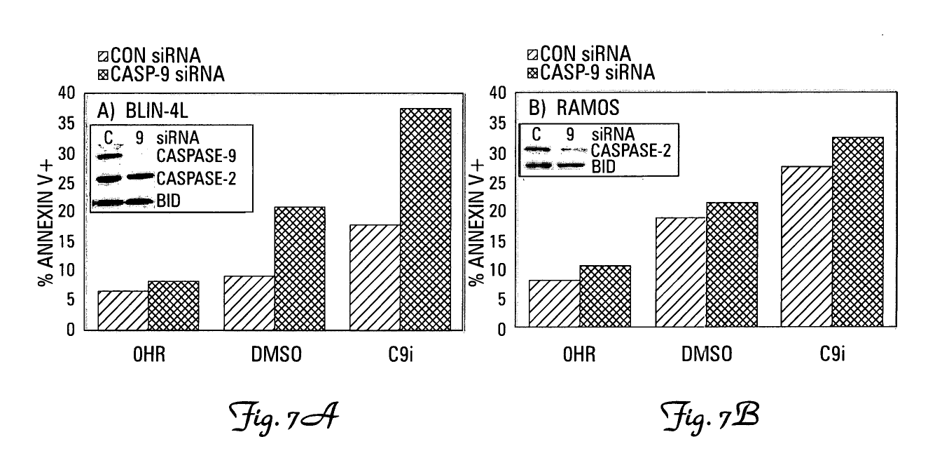

The inventors performed further studies to determine whether caspase-9 can be completely knocked out by means of RNA interference (RNAi). When siRNA molecules specific for caspase-9 were introduced into cells, no caspase-9 was seen when tested on a Western blot (FIG. 7). The inventors discovered that C9i induces apoptosis in caspase-9-deficient cells. BLIN-4L (FIG. 7A) and RAMOS (FIG. 7B) cells were electroporated with 8 μM SMARTpool (Dharmacon Inc.) caspase-9 siRNA or control siRNA and allowed to recover for 3 days in X-VIVO/10% FCS supplemented with 5 ng/ml IL-7 (BLIN-4L), or RPMI/10% FCS (RAMOS). After 3 days, the electroporated populations were assayed for Annexin V binding (to determine background levels of apoptosis), and then cultured in DMSO or 50 μM C9i for 8 hours. The cells were then harvested, stained with Annexin V, and analyzed by flow cytometry. Western blots showed the efficacy of caspase-9 reduction in caspase-9 siRNA(9)-treated cells compared to control siRNA(C)-treated cells. Thus, no caspase-9 was present at all in the cells. However, the inventors observed that in these cells, C9i still induced apoptosis. Thus, C9i clearly is acting on another molecule in the cell besides just caspase-9. Also, one can conclude that caspase-9 is not necessary for C9i-induced apoptosis to occur.

Normally, Z-LEHD-fmk binds to the active site of the caspase, and inhibits its activity. The present inventors, however, believe that it is also binding to another molecule, and it is somehow having an effect on the endoplasmic reticulum (ER).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for inducing apoptosis in a tumor cell comprising, contacting the tumor cell with an apoptosis-inducing molecule comprising a tetrapeptide moiety having an amino acid sequence numbered P4-P3-P2-P1, wherein P2 is a histidine residue, and wherein the tetrapeptide is a proapoptotic molecule.

2. The method of claim 1, wherein the apoptosis-inducing molecule further comprises at least one of a permeability-enhancing moiety and a cross-linking moiety.

3. The method of claim 1, wherein in the amino acid sequence P4-P3-P2-P1, P1 is an aspartic acid residue, P2 is a histidine residue, P3 is a glutamic acid residue and P4 is a leucine or tryptophan residue.

4. The method of claim 1, wherein the tetrapeptide moiety is Leu-Glu-His-Asp (SEQ ID NO:1).

5. The method of claim 1, wherein the tetrapeptide moiety is Trp-Glu-His-Asp (SEQ ID NO:10).

6. The method of claim 2, wherein the permeability-enhancing moiety is a benzoxycarbonyl group.

7. The method of claim 2, wherein the cross-linking moiety is fluoromethyl ketone.

8. The method of claim 1, wherein the apoptosis-inducing molecule is benzoxycarbonyl-LEHD-fluoromethyl ketone (SEQ ID NO 1).

9. The method of claim 1, wherein the tumor cells lack caspase-9 activity.

10. The method of claim 1, wherein the apoptosis is induced independently of other death stimuli.

11. A method for inducing apoptosis in a tumor cell comprising, contacting the tumor cell with a caspase-9 inhibitor.

12. A pharmaceutical composition comprising a pharmaceutically acceptable carrier and as active ingredient the apoptosis-inducing molecule of claim 1.

13. A method of treatment of leukemia or lymphoma in a patient, which method comprises administering to a patient a therapeutically effective amount of the apoptosis-inducing molecule of claim 1.


SEQUENCE LISTING (18 sequences)

SEQ ID NO: 1 - protein, 4 aa, Artificial Sequence
  Description of Artificial Sequence Synthetic peptide
LEHD

SEQ ID NO: 2 - protein, 4 aa, Artificial Sequence
  Description of Artificial Sequence Synthetic peptide
AEHD

SEQ ID NO: 3 - protein, 4 aa, Artificial Sequence
  Description of Artificial Sequence Synthetic peptide
LEAA

SEQ ID NO: 4 - protein, 4 aa, Artificial Sequence
  Description of Artificial Sequence Synthetic peptide
LEHA

SEQ ID NO: 5 - protein, 4 aa, Artificial Sequence
  Description of Artificial Sequence Synthetic peptide
LEED

SEQ ID NO: 6 - protein, 4 aa, Artificial Sequence
  Description of Artificial Sequence Synthetic peptide
LEAD

SEQ ID NO: 7 - protein, 4 aa, Artificial Sequence
  Description of Artificial Sequence Synthetic peptide
LEKD

SEQ ID NO: 8 - protein, 4 aa, Artificial Sequence
  Description of Artificial Sequence Synthetic peptide
LERD

SEQ ID NO: 9 - protein, 4 aa, Artificial Sequence
  Description of Artificial Sequence Synthetic peptide
LAHD

SEQ ID NO: 10 - protein, 4 aa, Artificial Sequence
  Description of Artificial Sequence Synthetic peptide
WEHD

SEQ ID NO: 11 - protein, 4 aa, Artificial Sequence
  Description of Artificial Sequence Synthetic peptide
AEHD

SEQ ID NO: 12 - protein, 4 aa, Artificial Sequence
  Description of Artificial Sequence Synthetic peptide
FEHD

SEQ ID NO: 13 - protein, 3 aa, Artificial Sequence
  Description of Artificial Sequence Synthetic peptide
EHD

SEQ ID NO: 14 - protein, 5 aa, Artificial Sequence
  Description of Artificial Sequence Synthetic peptide
VDVAD

SEQ ID NO: 15 - protein, 4 aa, Artificial Sequence
  Description of Artificial Sequence Synthetic peptide
DEVD

SEQ ID NO: 16 - protein, 4 aa, Artificial Sequence
  Description of Artificial Sequence Synthetic peptide
DQMD

SEQ ID NO: 17 - protein, 4 aa, Artificial Sequence
  Description of Artificial Sequence Synthetic peptide
VEID

SEQ ID NO: 18 - protein, 4 aa, Artificial Sequence
  Description of Artificial Sequence Synthetic peptide
IETD